(19) United States
(12) Patent Application Publication
(10) Pub. No.: US 2020/0024591 A1
(43) Pub. Date: Jan. 23, 2020

(54) BIOLOGICAL FLUIDS

(71) Applicant: Hewlett-Packard Development Company, L.P., Spring, TX (US)

(72) Inventors: Michael J. Day, Corvallis, OR (US); Greg Scott LONG, Corvallis, OR (US)

(73) Assignee: Hewlett-Packard Development Company, L.P., Spring, TX (US)

(21) Appl. No.: 16/500,721

(22) PCT Filed: Oct. 13, 2017
(86) PCT No.: PCT/US2017/056594

§ 371 Date: Oct. 3, 2019

(51) Int. Cl. C12N 9/96; C12N 9/24; G01N 1/38; B01L 3/02

(52) U.S. Cl. CPC C12N 9/96; C12N 9/2402; C12Y 302/01023; G01N 2001/386; B01L 3/0268; B01L 2400/0442; G01N 1/38

(57) ABSTRACT

The present disclosure is drawn to a biological fluid, including water, from 0.05 wt % to 3 wt % protein having an acidic isoelectric point (pI) less than about 6.5, and from 0.5 wt % to 20 wt % ionic protein stabilizer system. The ionic protein stabilizer system can include a buffer pair of a weak acid and a weak base, and a lyotropic series ionic compound.

### BACKGROUND

In the life sciences, there is utility in providing technologies for dispensing proteins, such as enzymes, DNA, RNA, antibodies, protein concentrates, or the like, onto various types of substrates. For example, proteins can be dispensed using a variety of technologies, including droppers or pipettes, MEMS or BioMEMS devices, fluid spotters, lab-on-chip devices, lateral flow reagent dispensers, microfluidic channeling and deposition devices, pneumatic devices, etc. Regardless of the technology used, each presents unique challenges associated with that particular deposition process.

### BRIEF DESCRIPTION OF THE DRAWING

FIG. 1 depicts an example lyotropic series with cationic and anionic lyotropic series ions shown on a left to right scale, wherein as ions move further to the left they tend to have one set of properties, and as ions move further to the right they tend to have another set of properties;

FIG. 2 depicts two different examples of lyotropic series ions coordinated with protein in water;

FIG. 3 depicts a schematic view of an example biological fluid ejection system in accordance with the present disclosure; and

FIG. 4 depicts an example method of ejecting a biological fluid in accordance with the present disclosure.

### DETAILED DESCRIPTION

In the life sciences, certain dispensing technologies for depositing proteins onto various types of substrates can provide certain advantages, such as precise deposition location, volumes, concentrations, etc. For example, enzymes, DNA, RNA, antibodies, protein concentrates, or the like, can be deposited on a biologically useful substrate using fluid-jetting technology. However, because of the general nature of proteins, some formulations and fluid-jetting approaches have been difficult because proteins can become degraded or deposited on various architecture used for deposition. This can be due, in part, to potential jetting temperatures and/or shear forces applied to the biological fluid during deposition or jetting. For example, it has been found that, particularly with respect to proteins having an acidic isoelectric point less than about 6.5, even moderately high temperatures and/or shear forces can lead to kogation and/or nozzle clogging. Kogation, in particular, can occur when the protein, or in some cases denatured protein, becomes deposited on the thermal resistor, eventually causing it to stop firing. By stabilizing the protein in the biological fluids of the present disclosure, proteins can become less likely to become denatured, and more likely to remain stable at thermal jetting temperatures, and furthermore, can become less likely to become deposited on thermal fluid-jet resistors, even if the protein does not become denatured.

To illustrate by way of example, by buffering proteins in solution, and in some cases, buffering to at or near the isoelectric point of the protein, the stability of the protein can be naturally increased, often with reduced protein precipitation. Furthermore, the inclusion of a lyotropic series compound, including one or both of a lyotropic series cation and/or a lyotropic series anion, proteins can be “salted in” or “salted out” to further stabilize them. In accordance with this, biological fluids can be prepared having a generally higher concentration of acidic isoelectric point (pI) protein, whereas without these added ingredients, these types of proteins may only be able to be thermally fluid-jetted at smaller quantities or at lower concentrations, depending on the specific protein. Furthermore, by stabilizing the protein using a buffer pair, and also further stabilizing the protein by salting-in or salting-out the protein using a lyotropic series compound, acidic pI proteins can be thermally jetted more reliably with reduced ejector jettability issues, such reduced as nozzle clogging, kogation, resistor buildup, ejector failure, etc. In many cases, the use of a buffer alone or a lyotropic series compound alone may not be enough to more than incrementally improve the ejectability of such proteins.

Thermally jetting of proteins, particularly proteins with an isoelectric point in the acidic pH range, e.g., defined herein as being less than about pH 6.5, for deposition on a substrate can be carried out with increased success. For purposes of the present disclosure, the “isoelectric point” or “pI” of a protein can be defined as the pH at which a protein surface has no net charge, e.g., exhibiting equal positive and negative charge. Thus, proteins in solution at a pH below the isoelectric point exhibit a net positive charge, and proteins in solution at a pH above their isoelectric point exhibit a net negative charge. Furthermore, the terms “acidic pI protein” or “acidic isolelectric point protein” or “protein having an acidic isoelectric point,” etc., refers to a protein having measured isoelectric point in a water-based fluid at 25° C. that is below about pH 6.5. It is also emphasized that “pI” refers to isoelectric point, whereas “pL” herein refers to picoliters, and these two terms should not be confused.

In accordance with this, in one example, a biological fluid can include water, from 0.05 wt % to 3 wt % protein having an acidic isoelectric point (pI) less than about 6.5, and from 0.5 wt % to 20 wt % ionic protein stabilizer system. The ionic protein stabilizer system can include a buffer pair of a weak acid and a weak base, and furthermore, can also include a lyotropic series ionic compound. In a specific example, the protein and the ionic protein stabilizer system can be present in the biological fluid at a weight ratio from 1:25 to 1:1. In another example, a concentration of the buffer pair and a weight ratio of the weak acid to the weak base can contribute to bringing the biological fluid to within 1 pH of the isoelectric point of the protein, or from 0.5 pH of the isoelectric point of the protein. Any of a number of buffer pairs can be used, such as monobasic sodium phosphate and dibasic sodium phosphate, monobasic potassium phosphate and dibasic potassium phosphate, citric acid and dibasic sodium phosphate, citric acid and dibasic potassium phosphate, citric acid and sodium citrate, citric acid and potassium citrate, acetic acid and sodium acetate, acetic acid and potassium acetate, ammonium chloride and ammonium hydroxide, thiocyanic acid and thiocyanate, or ammonium sulfate and trimethylamine n-oxide. The lyotropic series ionic compound can be any compound with a lyotropic series cation and/or lyotropic series anion described herein, but in one example, the lyotropic series cation can be (CH₃)₄N⁺, (CH₃)₂NH₂⁺, NH₄⁺, K⁺, or Na⁺; and/or the lyotropic series anion can be PO₄³⁻, SO₄²⁻, HPO₄²⁻, acetate⁻, or citrate⁻. There are other lyotropic series ions that can be used, but these specific lyotropic series ions can be particularly useful for use with acidic pI proteins. In another example, the lyotropic series ionic compound can include glycine, trimethyl amine-N-oxide, or betaine. In one specific example, relatively high concentrations of protein in a biological fluid suitable for thermal fluid-jet applications may tend to cause greater levels clogging and/or kogation issues compared to fluids with lower concentrations of protein. Thus, in a specific example, the protein can be present in the biological fluid at from 0.5 wt % to 3 wt %, the ionic protein stabilizer system can be present in the biological fluid at from 1 wt % to 15 wt %, and the protein and the ionic protein stabilizer system can be present in the biological fluid at a weight ratio from 1:15 to 1.1.

In another example, a biological fluid ejection system can include a biological fluid with water, from 0.05 wt % to 3 wt % protein having an acidic isoelectric point (pI) less than about 6.5, and from 0.5 wt % to 20 wt % ionic protein stabilizer system. The ionic protein stabilizer system can include a buffer pair of a weak acid and a weak base as well as a lyotropic series ionic compound. The system can further include a fluid reservoir for containing the biological fluid, and an ejector fluidly coupled to the fluid reservoir for thermally jetting the biological fluid received from the fluid reservoir. In this example, the ejector can operate at a temperature within a range from about 25° C. up to about 80° C., or more typically from about 40° C. up to about 60° C., and which generates a drop weight from 3 pL to 500 pL, or more typically from 8 pL to 40 pL. In further detail, a concentration of the buffer pair and a weight ratio of the weak acid to the weak base can contribute to bringing the biological fluid to within 1 pH (or within 0.5 pH) of the isoelectric point of the protein. As relatively high concentrations of protein in a biological fluid suitable for thermal fluid-jet applications can cause greater levels clogging and/or kogation compared to fluids with lower concentrations of protein, in one example, the biological fluid can include from 0.5 wt % to 3 wt % protein and from 1 wt % to 15 wt % ionic protein stabilizer system, and the protein and the ionic protein stabilizer system can be present in the biological fluid at a weight ratio from 1:15 to 1:1. In further detail, the system can include a substrate for receiving the thermally jetted biological fluid from the ejector. The substrate can be, for example, a well plate, a slide, a gel, a biochip, cellular culture, a vial, a tube, or a microarray.

In another example, a method of preparing a biological fluid can include combining a protein having an acidic isoelectric point (pI) less than about 6.5 with an ionic protein stabilizer system in water, wherein upon preparation, the biological fluid includes from 0.05 wt % to 3 wt % of the protein and from 0.5 wt % to 20 wt % of the ionic protein stabilizer system. The ionic protein stabilizer system can include, for example, a buffer pair of a weak acid and a weak base as well as a lyotropic series ionic compound. In one specific example, the protein and the ionic protein stabilizer system can be present in the biological fluid at a weight ratio from 1:25 to 1:1. In another example, a concentration of the buffer pair and a weight ratio of the weak acid to the weak base can contribute to bringing the biological fluid to within 1 pH (or within 0.5 pH) of the isoelectric point of the protein.

It is noted that when discussing the biological fluid, the biological fluid jetting system, or the method of preparing the biological fluid, each of these discussions can be considered applicable to other examples whether or not they are explicitly discussed in the context of that example unless expressly indicated otherwise. Thus, for example, when discussing a lyotropic series ionic compound related to the biological fluid, such disclosure is also relevant to and directly supported in context of various methods, and vice versa. Furthermore, for simplicity and illustrative purposes, the present disclosure is described by referring mainly to an example thereof. In the following description, numerous specific details are set forth in order to provide a thorough understanding of the present disclosure. It will be readily apparent however, that the present disclosure can be practiced without limitation to some of these specific details. In other instances, certain methods, compounds, compositions, and structures have not been described in detail so as not to unnecessarily obscure the present disclosure.

Proteins generally having an acidic isoelectric point at acidic pH levels can be particularly problematic when using thermal fluid-jet technology to deposit the proteins on a substrate. Though the present disclosure can provide benefits related to the ejection deposition of proteins within protein concentration ranges 0.05 wt % to 3 wt %, thermal fluid-jet resisters can often have even more difficulty at relatively higher concentrations, e.g., from about 0.5 wt % to about 3 wt % in solution. Thus, in accordance with examples of the present disclosure, by adding from 0.5 wt % to 20 wt % of an ionic protein stabilizer system to the protein-containing biological fluid, the protein can be stabilized for thermal fluid-jet application with typically reduced thermal fluid-jet ejection issues, such as reduced kogation and/or reduced nozzle clogging. In examples of the present disclosure, the protein and the ionic protein stabilizer system can be present in the biological fluid at a weight ratio from 1:25 to 1:1, from 1:20 to 1:1, from 1:15 to 1:1, from 1:20 to 1:2, from 1:15 to 1:4, from 1:25 to 1:2, or from 1:25 to 1:4, for example. The ionic protein stabilizer system in the present disclosure can be defined as the solids content of a buffer pair (weak acid and a weak base) and the solids content of a lyotropic series ionic compound as a whole (which includes at least one lyotropic series ion, but can include two lyotropic series ions).

It is noted that the present disclosure can be relevant to any of a number of proteins that have an isoelectric point (pI) with the acidic pH range. For illustrative purposes, Bovine Serum Albumin (BSA) is primarily described herein by way of example to illustrate certain advantages of the present disclosure, but other proteins having an acidic isoelectric point in the acidic pH range can also be used and can also show improvement with respect to thermal fluid-jet ejection properties, such as improved nozzle health, protein resistor deposition, and/or kogation. Example proteins that can be used having an acidic isoelectric point at an acidic pH include beta galactosidase (pI ˜4.6), BSA (pI ˜4.7), HDAC3 (pI ˜5), plasma amine oxidase (pI ˜5), HDAC 1 (pI ˜5.3), human IgM (˜5.5), MAP2K3 (pI ˜5.9), alcohol dehydrogenase (pI ˜6.2), or RPS6KA3 (pI ˜6.4), etc. Others can include various enzymes, DNA, RNA, antibodies, protein concentrates, etc. As a note, when isoelectric point is described herein with respect to a protein, the isoelectric point for purposes of the present disclosure is based on a measured (not calculated) isoelectric point under standard conditions, i.e. 25° C. in water, even though higher temperatures may be applied to the protein during thermal fluid-jet application processes.

As mentioned, the ionic protein stabilizer system can include a buffer pair of a weak acid and a weak base. The protein stabilizers system can also include a lyotropic series ionic compound. With specific reference to the buffer pair, suitable examples can include monobasic sodium phosphate and dibasic sodium phosphate, monobasic potassium phosphate and dibasic potassium phosphate, citric acid and dibasic sodium phosphate, citric acid and dibasic potassium phosphate, citric acid and sodium citrate, citric acid and potassium citrate, acetic acid and sodium acetate, acetic acid and potassium acetate, ammonium chloride and ammonium hydroxide, thiocyanic acid and thiocyanate, or ammonium sulfate and trimethylamine n-oxide. These specific buffer pairs are provided herein by example only, and other buffer pair systems can likewise be used, provided they do not act to denature or destroy the basic structure of the protein that is being ejected for deposition.

In certain specific examples, it has been found that the buffer pair used to prepare the biological fluid formulations of the present disclosure can be designed to bring the pH of the biological solution (containing the protein) to within 1 pH of the isoelectric point of the protein. In other examples, the buffer pair used to prepare the biological fluid formulations can be designed to bring the pH of the biological solution (containing the protein) to within 0.5 pH of the isoelectric point of the protein. In this manner, the protein can be brought nearer its isoelectric point, and thus, can be less strongly ionic than when outside of these ranges. This type of buffering can often have an even more positive impact on ejectability from thermal fluid-jet architecture, including nozzles and thermal fluid-jet resisters, compared to buffering to various more conventional biological system pH levels. For example, in some instances, protein (or degraded) buildup on the resister can be minimized, and more biological fluid nozzle throughput can occur as a result of modifying the pH of the biological fluid to near the protein isoelectric point. In further detail, if the pH is not brought to within 1 pH unit of the isoelectric point, some improvement can be achieved by buffering the biological solution in a direction toward the isoelectric point of the acidic pI protein, for example.

To illustrate further issues related to thermal fluid-jet ejection of proteins and improvements that can be achieved using the ionic protein stabilizer system described herein, a common thermal fluid jet resistor that fires at from about 35° C. to about 65 C, e.g., tantalum oxide surface, can be considered. Tantalum oxide has an isoelectric point (pI) from about 2.7 to 3. In the absence of anything else absorbed in or adsorbed on the surface thereof, such as a passivation layer, at pH 7.5, the Ta—O⁻ species is predominantly present (and the Ta—OH⁺ species minimized). Thus, a protein buffered at or even near its isoelectric point may typically have less affinity for the tantalum oxide resistor surface due to a more (net) neutral surface charge on the protein, thus reducing protein residue forming on the thermal resistor of the ejector.

When selecting a buffer solution to use, in some instances, the weak acid can be selected that has a pKₐ that is relatively close to the pH of the solution that is being sought, which in some examples may be within 1 pH or 0.5 pH of the isoelectric point of the protein. An advantage to selecting a weak acid relatively near the pH of the solution being sought is that it can provide for systems with relatively similar concentrations of the weak acid and the weak base (providing the buffer pair system with more neutralizing power either with respect to H⁺ and OH⁻). For example, by combining substances with pKₐ values differing by only two or less and adjusting the pH, a wide range of buffers can be used. Thus, in one example, citric acid can be used effectively with some of the acidic pI proteins of the present disclosure. Likewise, a monobasic (sodium or potassium) phosphate can also be a useful weak acid for use in accordance with examples of the present disclosure. Either of these weak acids can be used with a dibasic (sodium or potassium) phosphate as the weak base. To obtain a desired pH target, which may correspond to a pH value within 1 pH (or 0.5 pH), or which may approach or move in a direction, of the isoelectric point of the protein, the following buffer pairs provide relative combinations that can be used for three different buffer pairs, ranging from about pH 5 to 8, or about pH 5.8 to 8. Other buffer pairs can also be used, which would have different combination ratios and practical pH buffer ranges.

TABLE 1
Weak Acid and Weak Base Relative Volume vs. pH

| 0.1M Citric Acid | | 0.2M Na₂HPO_{4(dibasic)} | | |
|---|---|---|---|---|
| (mL) | | (mL) | | Target pH |
| 48.50 | mL | 51.50 | mL | 5 |
| 36.85 | mL | 63.15 | mL | 6 |
| 17.65 | mL | 82.35 | mL | 7 |
| 2.75 | mL | 97.25 | mL | 8 |

TABLE 2
Weak Acid and Weak Base Relative Concentration vs. pH

| KH₂PO_{4(monobasic)} | K₂HPO_{4(dibasic)} | |
|---|---|---|
| (wt %) | (wt %) | Target pH |
| 91.5 | 8.5 | 5.8 |
| 86.8 | 13.2 | 6 |
| 80.8 | 19.2 | 6.2 |
| 72.2 | 27.8 | 6.4 |
| 61.9 | 38.1 | 6.6 |
| 50.3 | 49.7 | 6.8 |
| 38.5 | 61.5 | 7 |
| 28.3 | 71.7 | 7.2 |
| 19.8 | 80.2 | 7.4 |
| 13.4 | 86.6 | 7.6 |
| 9.2 | 90.8 | 7.8 |
| 6 | 94 | 8 |

TABLE 3
Weak Acid and Weak Base Relative Concentration vs. pH

| NaH₂PO_{4(monobasic)} | Na₂HPO_{4(dibasic)} | |
|---|---|---|
| (wt %) | (wt %) | Target pH |
| 92 | 8 | 5.8 |
| 87.7 | 12.3 | 6 |
| 81.5 | 18.5 | 6.2 |
| 77.5 | 26.5 | 6.4 |
| 62.5 | 37.5 | 6.6 |
| 51 | 49 | 6.8 |
| 39 | 61 | 7 |
| 28 | 72 | 7.2 |
| 81 | 19 | 7.4 |
| 87 | 13 | 7.6 |
| 91.5 | 8.5 | 7.8 |
| 94.7 | 5.3 | 8 |

Even though there are examples where the pH can be brought to within 1 pH or 0.5 pH of the isoelectric point of the protein, and the proteins of the present disclosure have an isoelectric point generally in the acidic range, there are examples where the combination of a buffer pair and a lyotropic series compound outside of the 1 pH isoelectric window may still provide improved performance, even though the mechanism may not be fully understood. Thus, in accordance with examples of the present disclosure, some target pH ranges for thermal fluid-jet ejection of proteins having an acidic isoelectric point at less than about pH 6.5 can be from about pH 4 to pH 9, pH 4 to pH 8, pH 4 to pH 7.5, pH 4 to pH 7, pH 5 to pH 9, pH 5 to pH 8, pH 5 to pH 7.5, pH 5 to pH 7, pH 6 to pH 9, pH 6 to pH 8, pH 6 to pH 7.5, pH 6 to pH 7, pH 7 to pH 8, pH 7.2 to pH 7.8, etc. It is noted that when referring to the pH being brought to “within 1 pH” of the isoelectric point of the protein, this range includes 1 pH unit greater than the isoelectric point or 1 pH unit less than the isoelectric point. Thus, by way of example, if the isoelectric point is. 5, then the range for values within 1 pH of the isoelectric point would be from pH 4 to pH 6. Likewise, the range for values within 0.5 pH of the isoelectric point would be from pH 4.5 to pH 5.5.

In some cases, it may be desirable to fluid-jet a protein at a pH level that is outside of the above-described 1 pH (or 0.5 pH) window of the protein's isoelectric point. For example, the pH may in some cases be limited based on the purpose for dispensing the protein. For example, proteins may be fluid-jet ejected for activities such as PCR, tissue staining, cell-based assays, enzyme assays, ELISA, or any of a number of purposes or to provide any of a number of tools. In those cases, moving the pH of the biological fluid toward the isoelectric point of the protein can provide some ejectability improvement, even if the pH is not very closely matched with the isoelectric point of the protein. In these cases, some buffering with the additional ejectability benefits provided by the lyotropic series compound can still provide an acceptable biological fluid for thermal fluid-jet ejection from an architecture.

Turning more specifically now to the lyotropic (Hofmeister) series compounds that can also be added as a component of the ionic protein stabilizer system, there are any of a number of compounds that can be included. For example, in the context of biological fluids with proteins, citrates can be particularly useful. Examples include sodium citrate, potassium citrate, or citric acid (at a pH where the citric acid is in the form of a citrate). In some examples, it has been found that, in some instances, a fluid-jet ejector can jet from 10 times to 200 times more biological fluid from a thermal fluid-jet ejector when using a buffered solution with a citrate lyotropic series compound, depending on the concentration and type of acidic pI protein, the pH of the biological fluid influenced by the buffer pair selection and concentration, and with an added citrate lyotropic series compound. In other examples, a lyotropic series compounds, such as ammonium sulfate or trimethyl amine can be selected for use. Notably, not every lyotropic series compound works equally well for each acidic pI protein, but generally, lyotropic series compounds in a buffered solution can improve thermal fluid-jet ejectability throughput because of reduced kogation related to resistor buildup and/or nozzle clogging/ejector failure.

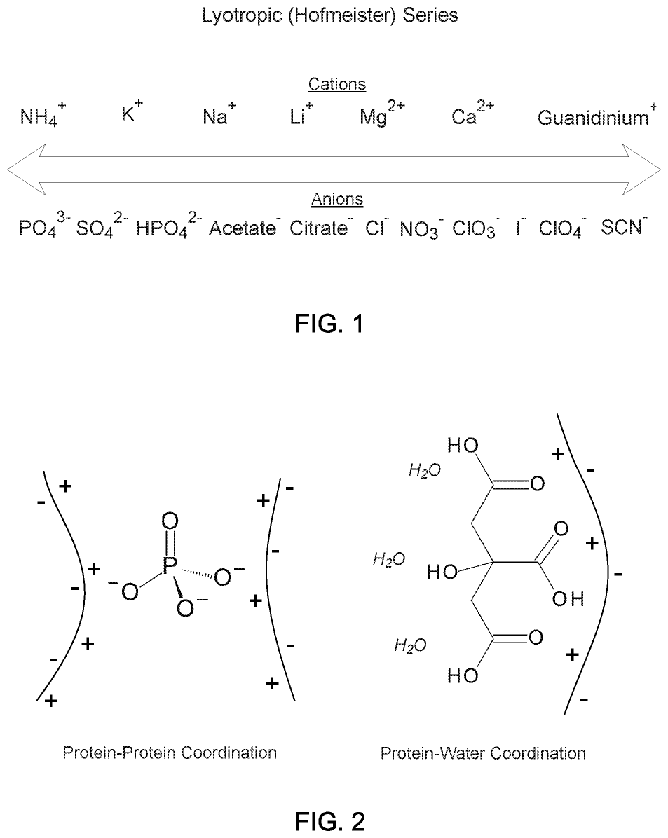

FIG. 1 provides an example of lyotropic series anions and cations that can be combined together, or which can be combined with other anions or cations (so that at least one of the two ions is a lyotropic series ion). In one example, however, both the anion and the cation can be lyotropic. In further detail in FIG. 1, as the ions (cations and anions) in the series move to the left, the ions tend to promote higher surface tension, have lower solubility in hydrocarbons, salt-out (precipitate and aggregate), have lower tendency for protein denaturation, exhibit greater protein stability, tend to be kosmotropic in that they increase the structure and stability of water, increase protein hydrophobic interactions, and increase protein-protein coordination with less water-protein coordination (see FIG. 2). These ions also can be included and carried to a point of precipitation of the protein. Notably, precipitated protein is still considered stable and not denatured, as the basic structure of the protein is preserved, even when precipitated. On the other hand, as the ions (cations and anions) in the series move to the right, the ions tend to promote lower surface tension, have higher solubility in hydrocarbons, salt-in (solubilize), have higher tendency for protein denaturation, exhibit lower protein stability, tend to be chaotropic in that they decrease the structure and stability of water, increase protein hydrophilic interactions, and increase water-protein coordination with less protein-protein coordination (see again FIG. 2). Notably, at certain concentrations, these components can be added to a point of irreversibly denaturing the protein in solution. Thus, a more careful addition of lyotropic series ions more toward the right side of the lyotropic series can be a consideration.

Notably, FIG. 1 is not an exhaustive list of all lyotropic series ions, but rather a representative list, provided in series, from a “salting out” left side to a “salting in” right side. More specifically, in addition to the lyotropic series compounds shown in FIG. 1, a more complete cationic series list (from left to right) can include (CH₃)₄N⁺, (CH₃)₂NH₂⁺, NH₄⁺, Rb⁺, K⁺, Na⁺, Cs⁺, Li⁺, Mg²⁺, Ca²⁺, Ba²⁺, and guanidium⁺ cations. Furthermore, a more complete anionic series list (from left to right) can include PO₄³⁻, SO₄²⁻, HPO₄²⁻, acetate⁻, citrate⁻, Cl⁻, Br⁻, NO₃⁻, ClO₃⁻, I⁻, ClO₄⁻, and SCN⁻. Not all of these lyotropic series ions can be particular suited for every type of protein, so selection can be carefully considered based on the selected protein, concentration of protein, presence of other ingredients, etc. For example, because some of these lyotropic series ions can be more favorably useful with proteins, e.g., ions more towards the left of the lyotropic series, in one specific example, the lyotropic series cations selected for use can be (CH₃)₄N⁺, (CH₃)₂NH₂⁺, NH₄⁺, K⁺, or Na⁺; and/or the lyotropic series anions selected for use can be PO₄³⁻, SO₄²⁻, HPO₄²⁻, acetate⁻, or citrate⁻, for example. Furthermore, there are other compounds that also have lyotropic properties that are not often listed with lyotropic series ions, e.g., zwitterionic compounds that have both a positive and negative charge that do not fit neatly into lyotropic series ion lists. For example, glycine, e.g., free glycine that is not part of the protein chain per se, trimethyl amine-N-oxide, or betaine can provide lyotropic-like stabilization to the proteins of the present disclosure. Thus, these compounds are defined herein to be lyotropic series compounds because they can behave like lyotropic series compounds. Furthermore, it is understood that they behave like lyotropic series ions generally found toward the left of the lyotropic series shown in FIG. 1, making them particularly suitable for protein stabilization in fluid-jettable biological fluids.

Generally, lyotropic series ions tend to “salt-out” nonpolar groups and “salt-in” peptide groups of a protein. The salting-out of nonpolar groups can be theorized using the cavity model. This model uses incremental surface tensions as a way of predicting observed values related to salting-out constants, within a factor of 3. The cavity model also predicts that salting-out typically increases with the number of carbon atoms found in aliphatic side chain of an amino acid. In further detail, nonspecific salting-in interaction can occur between simple ions and dipolar molecules. Ionic strength, rather than position along the lyotropic series, can also impact salting-in interactions. Whatever the mechanism, in general, lyotropic series compounds can be used to stabilize proteins for thermal fluid-jet ejection, particularly proteins having an acidic isoelectric point within the acidic range of less than about 6.5. These proteins can interact with lyotropic series ions in various ways, but at the same time, can also be susceptible to charge interactions. Thus, by buffering the proteins and including the lyotropic series compounds of the present disclosure, the proteins can be further stabilized to improve their thermal fluid-jettability properties.

In further detail, FIG. 2 depicts two examples of various types of stabilization that can occur when adding a lyotropic series compound to the biological fluids of the present disclosure. Thus, by way of example, using a phosphate ion, which is a lyotropic series anion on the far left of the lyotropic series shown in FIG. 1, in one example, a protein-protein coordination may occur to stabilize the proteins for thermal ejection from a jetting architecture. On the other hand, using a citrate ion (which is shown in this example for convenience as citric acid, but could be a mono-, di-, or tri-monovalent cation citrate, e.g., sodium citrate, dipotassium citrate, ammonium citrate, etc.) can result a protein-water coordination that has a stabilizing effect on the protein for ejection as well. Citrate is more towards the center of the lyotropic series compared to the phosphate, and thus, more protein-water coordination may occur, but it is understood that citrate can also form protein-protein coordination as well. Thus, the lyotropic additive can interact with the protein directly, such as by creating more hydrogen bonding around the protein to keep it hydrated, and thus stabilized, or the lyotropic series compound can modify the general molecular order of the water, e.g., creating more or less order, resulting in enhanced protein stabilization. Regardless the mechanism, whether more protein-protein coordination, more protein-water coordination, more salting-in, more salting-out, etc., the use of lyotropic series ions or compounds from the lyotropic series can have a stabilizing effect on proteins. Furthermore, the stabilizing effect can be particularly useful for proteins having an isoelectric point within the acidic pH range. Though phosphate ion and citrate ion (from citric acid) are shown in FIG. 2, other suitable lyotropic series ions that can be used with similar effect include ammonium compounds, sulfate compounds, or in one specific example, ammonium sulfate, among others.

In further detail regarding the addition of lyotropic series compounds and providing improved thermal fluid-jet stability, many proteins can under certain conditions form salt bridges from anionic carboxylate groups such as can be present in aspartic acid or glatamic acid, from cationic ammonium that can be present in lysine, or from cationic guanidinium that can be present in arginine. Other amino acids that have ionic side chains, such as histidine, tyrosine, serine, etc., can also participate in forming salt bridges, depending on outside factors that impact their pKₐ, for example. Salt bridges, defined as having a distance less than about 4 angstroms, can thus be impacted with respect to stability based on the pH level of the fluid in which the protein is contained. In accordance with examples of the present disclosure, a lyotropic series compound, such as a citric acid salt (or citrate), can ameliorate or reduce intra-protein salt bridging, thus increasing the solubility of the protein. In this state, the protein can be “modified” (but not denatured) sufficiently to undergo thermal fluid-jet stresses without thermal or shear induced precipitation that can otherwise occur, which can lead to unwanted protein deposition on the thermal resistor. Thus, this is an example of how the biological fluid components, when used together as described herein, can be sufficiently complicated that all of the mechanisms may not be fully understood. However, this combination of components can provide proteins at a usable pH levels that are not denatured, even upon ejecting from thermal fluid-jet architecture.

By way of specific example, 2 wt % of a protein, such as BSA (pI ˜4.7), can be formulated in a buffer solution, e.g., about 40 to about mMolar, with 500 mMolar of a sodium citrate or ammonium sulfate, providing acceptable stabilization of the protein, whereas without the buffer and the lyotropic series compound, the BSA protein would quickly become deposited on a thermal resistor and/or clog nozzles of a thermal fluid-jet ejector. Thus, typically, with higher protein concentrations comes a higher probably of thermal fluid-jet ejector failures, with more problems occurring at from 0.1 wt % to 3 wt % protein, or even more particularly from 0.5 wt % to 3 wt % protein, with many complete failures occurring quickly above about 1 wt %. In examples of the present disclosure, the lyotropic series compound can be added at a concentration of about 4 to 15 times by weight greater than a concentration of the buffer pair, though this range is not intended to be limiting.

Once the biological fluid is formulated and ready for thermal fluid-jet ejection, such deposition process can occur at temperatures as low as about 25° C. up to about 80° C., and more typically from 35° C. to 65° C., or from 40° C. to 60° C. Notably, fluids that are closer to the thermal resistor are more likely to be exposed to higher temperatures within this range, and thus, this may explain why some protein denaturing and thermal resistor deposition may occur, particularly without inclusion of the ionic protein stabilizer system of the present disclosure. Furthermore, when thermally ejecting fluid from an architecture, dispensing of droplet volumes can range from just a few picoliters up to several mL (e.g., 3 pL to 4 mL, 3 pL to 1 mL, 3 pL to 1 μm, 3 pL to 1 nL, 3 pL to 500 pL, 3 pL to 100 pL, 3 pL to 50 pL, 3 pL to 20 pL, 3 pL to 10 pL, 8 pL to 4 mL, 8 pL to 1 mL, 8 pL to 1 μm, 8 pL to 1 nL, 8 pL to 500 pL, 8 pL to 100 pL, 8 pL to 50 pL, 8 pL to 40 pL, 8 pL to 33 pL, etc.). However, there can be particular interest in ejection volumes from about 3 pL to about 500 pL in some examples. Notably, smaller ejection nozzles or orifices tend to create more pronounced jettability problems, so ejection at the smaller end of the can of be problematic. Thus, in one example of the present disclosure, fluid-jet ejection can be carried out using droplet sizes ranging from 3 pL to 500 pL, from 3 pL to 100 pL, from 3 pL to 50 pL, 3 pL to 20 pL, from 3 pL to 10 pL, from 8 pL to 500 pL, from 8 pL to 100 pL, from 8 pL to 50 pL, from 8 pL to 40 pL, or from 8 pL to 33 pL, for example.

In addition to the water, the protein, and the components of the ionic protein stabilizer system, other ingredients can be added, such as a surfactant, a dye, a fluorescent dye, nano-particles, cell growth media components, polymers, surfactant, assay components, ATP, and/or NAD, for example. These can be added at concentrations that do not substantially denature the protein, and/or can be added for any of a number of purposes suitable for deposition and or use of deposited proteins on any of a number of substrates.

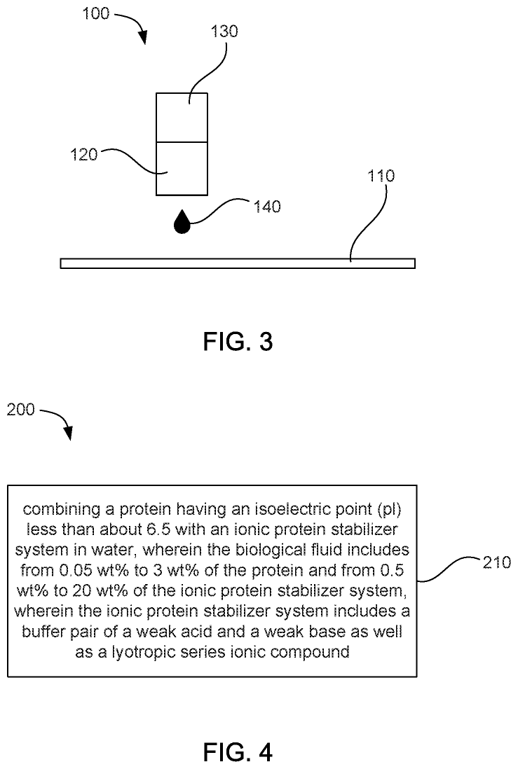

As mentioned, the present disclosure is also drawn to a biological fluid ejection system 100 for ejecting or jetting a biological fluid 140, as shown in FIG. 3. The biological fluid can include water, from 0.05 wt % to 3 wt % protein having an acidic isoelectric point (pI) less than about 6.5, and from 0.5 wt % to 20 wt % ionic protein stabilizer system. The ionic protein stabilizer system can include a buffer pair of a weak acid and a weak base as well as a lyotropic series ionic compound. The system can also include a fluid reservoir 130 for containing the biological fluid, and an ejector 120 fluidly coupled to the fluid reservoir for thermally jetting the biological fluid received from the fluid reservoir. Any of the details described above that relate to the biological fluid can be relevant to the biological fluid ejection system shown in FIG. 3. In further detail, the ejector can operate at a temperature within a range from about 25° C. up to about 80° C. In another specific example, the ejector can be adapted to eject a drop weight from 3 pL to 500 pL. A concentration of the buffer pair and a weight ratio of the weak acid to the weak base can contribute to bringing the biological fluid to within 1 pH of the isoelectric point of the protein. In one example, the system can further include a substrate 110 for receiving the ejected biological fluid, such as a well plate, a slide, a gel, a biochip, cellular culture, a vial, a dish, a tube, or a microarray.

The present disclosure also sets for a method 200 of preparing biological fluids, as shown in FIG. 4. In one example, the method can include combining 210 a protein having an acidic isoelectric point (pI) less than about 6.5 with an ionic protein stabilizer system in water, wherein the biological fluid includes from 0.05 wt % to 3 wt % of the protein and from 0.5 wt % to 20 wt % of the ionic protein stabilizer system. The ionic protein stabilizer system can include a buffer pair of a weak acid and a weak base as well as a lyotropic series ionic compound. Any of the details described above that relate to the biological fluid can be relevant to the present method.

It is noted that, as used in this specification and the appended claims, the singular forms “a,” “an,” and “the” include plural referents unless the content clearly dictates otherwise.

As used herein, the term “about” is used to provide flexibility to a numerical range endpoint by providing that a given value may be “a little above” or “a little below” the endpoint. The degree of flexibility of this term can be dictated by the particular variable and would be within the knowledge of those skilled in the art to determine based on experience and the associated description herein.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight ratio range of 1 wt % to 20 wt % should be interpreted to include not only the explicitly recited limits of 1 wt % and 20 wt %, but also to include individual weights such as 2 wt %, 11 wt %, 14 wt %, and sub-ranges such as 10 wt % to 20 wt %, 5 wt % to 15 wt %, etc.

### EXAMPLES

The following examples illustrate the technology of the present disclosure. However, it is to be understood that the following is only exemplary or illustrative of the application of the principles of the presented formulations and methods. Numerous modifications and alternative methods may be devised by those skilled in the art without departing from the spirit and scope of the present disclosure. The appended claims are intended to cover such modifications and arrangements. Thus, while the technology has been described above with particularity, the following examples provide further detail in connection with what are presently deemed to be certain acceptable examples.

### Example 1

Various biological fluids containing various proteins were ejected from a thermal fluid-jet ejector at a resister temperature ranging from about 30° C. to 45° C., and having a drop weigh of about 30 pL. The protein concentration in the various the biological fluids were present at or near about 0.5 wt % in water. The formulation also included 0.25 wt % of a surfactant and the formulation as a whole was buffered using a phosphate buffered saline (PBS) system (pH 7.8) to determine if a mildly basic solution including moderate concentrations of various proteins could be effectively jetted from a thermal fluid-jet ejector. Table 3 below provides the various proteins tested and the approximate isoelectric point for each protein, as well as whether or not the protein left a residue on the tantalum oxide thermal fluid-jet resistor, as follow:

TABLE 3

| Protein | Isoelectric Point (pl) | Residue on Resistor |
|---|---|---|
| Beta Galactosidase | 4.61 | Yes |
| BSA | 4.7 | Yes** |
| HDAC 3 | 4.98 | Yes |
| HDAC 1 | 5.3 | Yes |
| MAP2K3 | 5.9 | Yes |
| RPS6KA3 | 6.41 | Yes** |
| MAP2K6 | 6.61 | No |
| MAPKAPK3 | 6.87 | No |
| IgG (mouse) | 7.2* | No |
| BRAF | 7.29 | No |
| IgM | 7.4* | No |
| Lysozyme | 9.32 | No |
| Trypsin | 10.5 | No** |

*Some question of actual pl
**BSA-heavy buildup; Rps6KA3-light buildup; and Trypsin-no buildup

Thus, as can be seen in Table 3, resistor residue was not particularly problematic when thermally ejecting proteins having an acidic isoelectric point above about 6.5.

### Example 2

Various biological fluids having acceptable properties for jetting from thermal fluid-jet architecture, such architecture including a tantalum oxide resistor fired at a resistor temperature about 40° C., can be prepared in accordance with Table 4, as follows:

TABLE 4

| | | Fluid 1 | | Fluid 2 | | Fluid 3 | |
|---|---|---|---|---|---|---|---|
| Biological Fluid | | (parts by | | (parts by | | (parts by | |
| Component | Type | weight) | | weight) | | weight) | |
| †Sodium phosphate | Weak Base | 130 | mg | 130 | mg | 130 | mg |
| dibasic | | | | | | | |
| Sodium phosphate | Weak Acid | 900 | mg | — | | — | |
| monobasic | | | | | | | |
| Citric Acid | Weak Acid | — | | 900 | mg | 900 | mg |
| Sodium citrate | Lyotropic | 12 | g | 12 | g | — | |
| dehydrate | Series | | | | | | |
| | Compound | | | | | | |
| Disodium citrate | Lyotropic | — | | — | | 12 | g |
| | Series | | | | | | |
| | Compound | | | | | | |
| BSA (4.7 pl) | Protein | 1 | g | 1 | g | 1 | g |
| Water | Vehicle | 100 | g | 100 | g | 100 | g |

†When sodium is used for the buffer components or the lyotropic series cation, potassium or ammonium, for example, can be substituted therewith. Sodium is used in this example for simplicity.

### Example 3

0.1 wt % HDAC 1 (pI 5.3) and 0.1 wt % HDAC 3 (pI 4.98) were each formulated and buffered in a water vehicle to pH 7.5 and to pH 5.6 using different ratios of the buffer pair Na₂HPO₄ (weak acid) and NaH₂PO₄ (weak base). When ejected using thermal fluid-jet architecture at a resister temperature ranging from about 30° C. to 45° C. (drop weight about 30 pL), residual build up at the resistor was observed at pH 7.5, whereas the resistor actuated at the same throughput volume at pH 5.6 (within pH 0.5 of pI for HDAC 1 and within pH 1 of pI for HDAC 3) was essentially clean. In further detail, by adding a citrate lyotropic series compound, higher protein concentrations, e.g., up to at least 1 wt %, and/or greater throughput volumes can be achieved.

### Example 4

Two samples of 1 wt % BSA (pI 4.7) were each formulated and buffered in a water vehicle to pH 7.5 and to pH 4.5 using buffer pairs including citric acid (weak acid) and NaH₂PO₄ (weak base). The biological fluid was then thermally jetted onto various well plates. When 31 nL×10 pL (31 nL dispensed into each well plate for a total of 10 pL) was jetted using a thermal fluid-jet ejector (resister temperature from 30° C. to 45° C.; pH 7.5 fluid drop weight 26 pL and cv 12%; and pH 4.6 fluid drop weight 28 pL and cv 4%), significant residual build up at the resistor was observed at pH 7.5, whereas the resistor actuated under the same conditions at pH 4.5 (within pH 0.5 of pL) was essentially clean. As a note, “cv” is the coefficient of variation, which is the ratio of the standard deviation compared to the mean. The same test was repeated, but this time at throughput volume 500 nL×200 μL. The biological fluid prepared at pH 7.5 clogged at 100 pL, whereas the biological fluid prepared at pH 4.5 jetted all of the fluid until the thermal fluid-jet ejector dry fired. In further detail, by adding citrate lyotropic series compound, higher protein concentrations, e.g., up to 3 wt %, and greater throughput volumes, e.g., up to at least about 1.5 mL, can be achievable

### Example 5

0.5 wt % BSA in water alone was jetted using a thermal fluid-jet ejector at 40° C. (fluid drop weight 30 pL). Only about 0.2 pL was dispensed before the protein build up caused the resistor to completely fail.

### Example 6

3 wt % BSA was formulated in a 50 mMolar of a sodium phosphate monobasic/sodium phosphate dibasic buffer at pH 7.8. The formulation was thermally jetted from a fluid-jet ejector at 40° C. (fluid drop weight 30 pL). After dispensing only 15 pL, the ejector failed. At 7 pL, prior to failing, significant resistor build up was photographically recorded.

### Example 7

To evaluate the benefit of combining a buffer pair (phosphate monobasic/sodium phosphate dibasic) with a lyotropic series compound, a biological fluid containing 1 wt % BSA, 50 mMolar of the phosphate buffer pair at 5.6 pH, and an ammonium sulfate lyotropic series compound was prepared. The biological fluid was thermally jetted using a fluid-jet ejection architecture with a resistor at 35° C. and the resistors remained clean after a significant volume of biological fluid was ejected (400 μL). In certain examples, the ammonium sulfate lyotropic series compound can be added at a concentration of about 4 to 15 times by weight greater than the concentration of the phosphate buffer pair.

While the present technology has been described with reference to certain examples, those skilled in the art will appreciate that various modifications, changes, omissions, and substitutions can be made without departing from the spirit of the disclosure. It is intended, therefore, that the disclosure be limited only by the scope of the following claims.

What is claimed is:

1. A biological fluid, comprising:
water;
from 0.05 wt % to 3 wt % protein having an acidic isoelectric point (pI) less than about 6.5; and
from 0.5 wt % to 20 wt % ionic protein stabilizer system, wherein the ionic protein stabilizer system includes:
a buffer pair of a weak acid and a weak base, and
a lyotropic series ionic compound.

2. The biological fluid of claim 1, wherein the protein and the ionic protein stabilizer system is present in the biological fluid at a weight ratio from 1:25 to 1:1.

3. The biological fluid of claim 1, wherein a concentration of the buffer pair and a weight ratio of the weak acid to the weak base contributes to bringing the biological fluid to within 1 pH of the isoelectric point of the protein.

4. The biological fluid of claim 1, wherein a concentration of the buffer pair and a weight ratio of the weak acid to the weak base contributes to bringing the biological fluid to within 0.5 pH of the isoelectric point of the protein.

5. The biological fluid of claim 1, wherein the buffer pair includes: monobasic sodium phosphate and dibasic sodium phosphate, monobasic potassium phosphate and dibasic potassium phosphate, citric acid and dibasic sodium phosphate, citric acid and dibasic potassium phosphate, citric acid and sodium citrate, citric acid and potassium citrate, acetic acid and sodium acetate, acetic acid and potassium acetate, ammonium chloride and ammonium hydroxide, thiocyanic acid and thiocyanate, or ammonium sulfate and trimethylamine n-oxide.

6. The biological fluid of claim 1, wherein the lyotropic series ionic compound includes a lyotropic series cation selected from (CH₃)₄N⁺, (CH₃)₂NH₂⁺, NH₄⁺, K⁺, or Na⁺; or a lyotropic series anion selected from PO₄³⁻, SO₄²⁻, HPO₄²⁻, acetate⁻, or citrate⁻.

7. The biological fluid of claim 6, wherein the lyotropic series ionic compound includes both the lyotropic series cation and the lyotropic series anion.

8. The biological fluid of claim 1, wherein the lyotropic series ionic compound includes glycine, trimethyl amine-N-oxide, or betaine.

9. The biological fluid of claim 1, wherein the protein is present in the biological fluid at from 0.5 wt % to 3 wt %, the ionic protein stabilizer system is present in the biological fluid at from 1 wt % to 15 wt %, and wherein the protein and the ionic protein stabilizer system are present in the biological fluid at a weight ratio from 1:15 to 1:1.

10. A biological fluid ejection system, comprising:
a biological fluid including water, from 0.05 wt % to 3 wt % protein having an acidic isoelectric point (pI) less than about 6.5, and from 0.5 wt % to 20 wt % ionic protein stabilizer system, wherein the ionic protein stabilizer system includes a buffer pair of a weak acid and a weak base as well as a lyotropic series ionic compound;
a fluid reservoir for containing the biological fluid; and
an ejector fluidly coupled to the fluid reservoir for thermally jetting the biological fluid received from the fluid reservoir.

11. The biological fluid ejection system of claim 10, wherein the ejector operates at a temperature within a range from about 25° C. up to about 80° C. and generates a drop weight from 3 pL to 500 pL.

12. The biological fluid ejection system of claim 10, wherein a concentration of the buffer pair and a weight ratio of the weak acid to the weak base contributes to bringing the biological fluid to within 1 pH of the isoelectric point of the protein.

13. The biological fluid ejection system of claim 10, further comprising a substrate for receiving the biological fluid thermally jetted from the ejector, the substrate selected from a well plate, a slide, a gel, a biochip, cellular culture, a vial, a dish, a tube, or a microarray.

14. A method of preparing a biological fluid, comprising combining a protein having an acidic isoelectric point (pI) less than about 6.5 with an ionic protein stabilizer system in water, wherein the biological fluid includes from 0.05 wt % to 3 wt % of the protein and from 0.5 wt % to 20 wt % of the ionic protein stabilizer system, wherein the ionic protein stabilizer system includes a buffer pair of a weak acid and a weak base as well as a lyotropic series ionic compound.

15. The method of claim 14, wherein the protein and the ionic protein stabilizer system are present in the biological fluid at a weight ratio from 1:25 to 1:1, and wherein a concentration of the buffer pair and a weight ratio of the weak acid to the weak base contributes to bringing the biological fluid to within 1 pH of the isoelectric point of the protein.
